(19) United States
(12) Patent Application Publication
(10) Pub. No.: US 2018/0265433 A1
(43) Pub. Date: Sep. 20, 2018

(54) MULTI-STAGE SELECTIVE HYDROGENATION PROCESS FOR PROCESSING OF BUTADIENE EXTRACTION UNIT

(71) Applicant: UOP LLC, Des Plaines, IL (US)

(72) Inventor: Steven Lee Krupa, Fox River Grove, IL (US)

(21) Appl. No.: 15/982,986

(22) Filed: May 17, 2018

Related U.S. Application Data
(63) Continuation-in-part of application No. PCT/US2016/065177, filed on Dec. 6, 2016.
(60) Provisional application No. 62/264,028, filed on Dec. 7, 2015.

(51) Int. Cl. C07C 11/167; C07C 7/167; C07C 7/11; C07C 7/04; C07C 5/09

(52) U.S. Cl. CPC C07C 11/167; C07C 7/167; C07C 5/09; C07C 7/04; C07C 7/11

(57) ABSTRACT

A process is present for increasing the yields of 1,3 butadiene. The process includes recovering 1,3 butadiene from a cracking unit that generates a crude C4 stream. The 1,3 butadiene is separated and the remaining C4 process stream components are further reacted and dehydrogenated to generate 1,3 butadiene in a subsequent process stream. The subsequent process stream is recycled to recover the additional 1,3 butadiene.

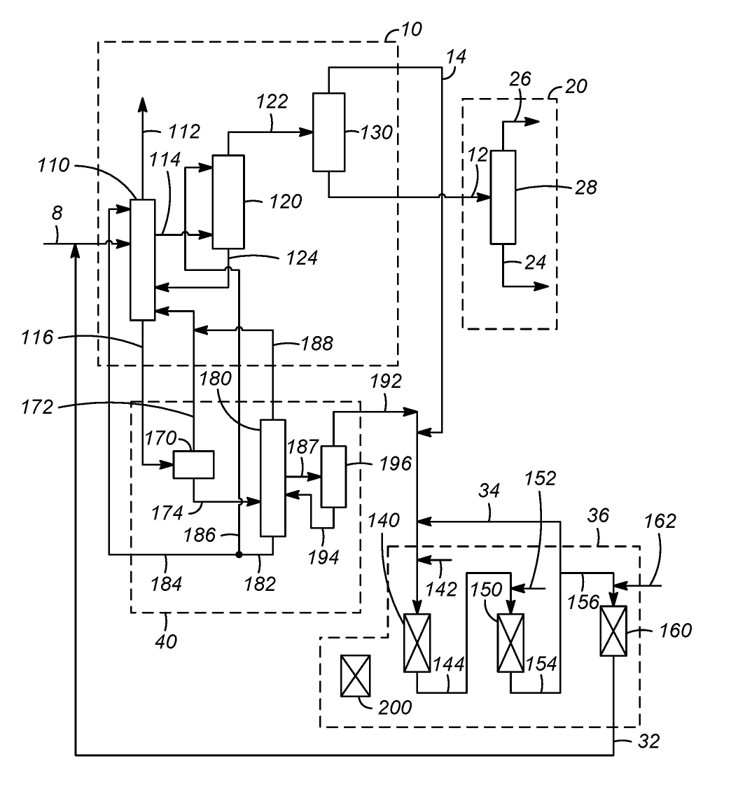

### CROSS-REFERENCE TO RELATED APPLICATIONS

This application is a Continuation-In-Part of copending International Application No. PCT/US2016/065177 filed Dec. 6, 2016, which application claims priority from U.S. Provisional Application No. 62/264,028 filed Dec. 7, 2015, now expired, the contents of which cited applications are hereby incorporated by reference in their entirety.

### FIELD OF THE INVENTION

The present invention relates to a process for the production of butadiene. In particular, this is a process for the improvement in the production and recovery of butadiene.

### BACKGROUND

The use of plastics and rubbers are widespread in today's world. The production of these plastics and rubbers are from the polymerization of monomers which are generally produced from petroleum. The monomers are generated by the breakdown of larger molecules to smaller molecules which can be modified. The monomers are then reacted to generate larger molecules comprising chains of the monomers. An important example of these monomers is light olefins, including ethylene and propylene, which represent a large portion of the worldwide demand in the petrochemical industry. Light olefins, and other monomers, are used in the production of numerous chemical products via polymerization, oligomerization, alkylation and other well-known chemical reactions. Producing large quantities of light olefin material in an economical manner, therefore, is a focus in the petrochemical industry. These monomers are essential building blocks for the modern petrochemical and chemical industries. The main source for these materials in present day refining is the steam cracking of petroleum feeds.

Another important monomer is butadiene. Butadiene is a basic chemical component for the production of a range of synthetic rubbers and polymers, as well as the production of precursor chemicals for the production of other polymers. Examples include homopolymerized products such as polybutadiene rubber (PBR), or copolymerized butadiene with other monomers, such as styrene and acrylonitrile. Butadiene is also used in the production of resins such as acrylonitrile butadiene styrene.

Butadiene is typically recovered as a byproduct from the cracking process, wherein the cracking process produces light olefins such as ethylene and propylene. With the increase in demand for rubbers and polymers having the desired properties of these rubbers, an aim to improving butadiene yields from materials in a petrochemical plant will improve the plant economics.

The demand for plastics such as polyethylene and polypropylene has increased substantially, and will continue to increase in the foreseeable future. Due to the increase demand, the increase in demand for the monomers, ethylene and propylene or light olefins, has also increased. This increase in demand has led to improvements in the processes for the production of light olefins. The improvements increase yields from traditional sources, such as naphtha cracking, and from other sources by diverting other hydrocarbon streams for the production of light olefins. For purposes of the present invention, the reference to steam cracking, as used hereinafter, is intended to include any cracking unit, which can be a catalytic cracker, a steam cracker, or a cracking unit for hydrocarbon sources other than naphtha. With increased availability of ethane from associated gas of crude oil production, as well as, from increased recovery of natural gas liquids (NGL) which contains large amounts of ethane, use of ethane as a feed to steam crackers has increased. When ethane is used as a feedstock to a steam cracker, the byproduct C4 stream is significantly reduced. As a result of these changes, the production of byproducts C4s from the cracking process has decreased. An important part of this byproduct is the recovery of butadiene, and through changes in feedstocks for light olefins production, butadiene recovery has been reduced, or is not keeping up with increased demand.

### SUMMARY

The present invention is a process for increasing the butadiene yields from a crude C4 stream.

The present invention is an integrated process for the production of butadiene, comprising passing a steam cracked process stream to a butadiene separation system, thereby generating a first butadiene stream and an acetylene stream; passing the acetylene stream to a selective hydrogenation reactor system, thereby generating a second butadiene stream and a recycle stream; and passing the first butadiene stream to a second butadiene separation system. An embodiment of the invention is one, any or all of prior embodiments in this paragraph up through the first embodiment in this paragraph wherein the second butadiene separation system comprises passing the first butadiene stream to a butadiene fractionation unit to generate a 1,3 butadiene overhead stream and a 1,2 butadiene bottoms stream. An embodiment of the invention is one, any or all of prior embodiments in this paragraph up through the first embodiment in this paragraph wherein the 1,2 butadiene bottoms stream comprises 1,2 butadiene and heavies. An embodiment of the invention is one, any or all of prior embodiments in this paragraph up through the first embodiment in this paragraph wherein the selective hydrogenation reactor system comprises passing the acetylene stream and a hydrogen stream to a first selective hydrogenation reactor to generate a first reactor effluent stream with reduced acetylenes; passing the first reactor effluent stream and a second hydrogen stream to a second selective hydrogenation reactor to generate a second reactor effluent stream with reduced acetylenes; and passing a first portion of the second reactor effluent stream and a third hydrogen stream to a third selective hydrogenation reactor to generate a third reactor butadiene stream with less than 100 ppmw acetylenes. If desired, much lower levels of acetylenes may be achieved. For example, a third reactor butadiene stream may have less than 50, less than 20, less than 10, or less than 5 wppm ethylacetylene. An embodiment of the invention is one, any or all of prior embodiments in this paragraph up through the first embodiment in this paragraph further comprising passing a second portion of the second reactor effluent stream to the first selective hydrogenation reactor. An embodiment of the invention is one, any or all of prior embodiments in this paragraph up through the first embodiment in this paragraph further comprising passing the third reactor butadiene stream to the butadiene separation system. An embodiment of the invention is one, any or all of prior embodiments in this paragraph up through the first embodiment in this paragraph wherein the steam cracked process stream comprises crude C4 hydrocarbons. An embodiment of the invention is one, any or all of prior embodiments in this paragraph up through the first embodiment in this paragraph wherein the crude C4 hydrocarbons include butenes, butanes, butadienes, ethylacetylene and vinylacetylene. An embodiment of the invention is one, any or all of prior embodiments in this paragraph up through the first embodiment in this paragraph wherein the butadiene separation system comprises passing the steam cracked process stream to a first absorption separation column, and passing a solvent to the first absorption separation column, to generate a raffinate stream comprising butenes and butanes; a first intermediate stream comprising butadienes, vinylacetylene and ethylacetylene; and a first bottoms stream comprising butadienes, vinylacetylene, ethylacetylene and solvent; passing the intermediate stream to a second absorption separation column, and passing a solvent to the second absorption separation column, to generate a second overhead stream comprising butadienes, methylacetylene and vinylacetylene and pentenes; and a second bottoms stream comprising acetylenes and solvent; and passing the second overhead stream to a first fractionation column to generate the first butadiene stream and an third overhead stream comprising methyl acetylene and butadiene. An embodiment of the invention is one, any or all of prior embodiments in this paragraph up through the first embodiment in this paragraph further comprising passing the third overhead stream to the selective hydrogenation reactor system. An embodiment of the invention is one, any or all of prior embodiments in this paragraph up through the first embodiment in this paragraph further comprising passing the second bottoms stream to the first absorption separation column. An embodiment of the invention is one, any or all of prior embodiments in this paragraph up through the first embodiment in this paragraph further comprising passing the first bottoms stream to a solvent recovery unit. An embodiment of the invention is one, any or all of prior embodiments in this paragraph up through the first embodiment in this paragraph wherein the solvent recovery unit comprises passing the first bottoms stream to a first flash separation tank to generate a vapor stream comprising butadienes and acetylenes, and a liquid stream comprising solvent and butadienes and acetylenes; passing the vapor stream to the first absorption separation column; passing the liquid stream to a first solvent fractionation column to generate a first solvent fractionation overhead stream; a first solvent fractionation bottoms stream and a first solvent fractionation intermediate stream; passing the first solvent fractionation intermediate stream to a second solvent fractionation column to generate a second solvent fractionation overhead stream comprising acetylenes, and a second solvent fractionation bottoms stream comprising solvent; and passing the second solvent fractionation overhead stream to the selective hydrogenation reactor system. An embodiment of the invention is one, any or all of prior embodiments in this paragraph up through the first embodiment in this paragraph further comprising passing a first portion of the first solvent fractionation bottoms stream to the first absorption separation column. An embodiment of the invention is one, any or all of prior embodiments in this paragraph up through the first embodiment in this paragraph further comprising passing a second portion of the first solvent fractionation bottoms stream to the second absorption separation column. An embodiment of the invention is one, any or all of prior embodiments in this paragraph up through the first embodiment in this paragraph wherein the solvent used in the adsorption separation columns is selected from the group consisting of n-methyl-2-pyrolidone (NMP), dimethylformamide (DMF), acetonitrile, and mixtures thereof.

A second embodiment of the invention is a process for the production of butadiene, comprising passing a steam cracked process stream to a butadiene separation system, thereby generating a first butadiene stream and a solvent stream; passing the solvent stream to a solvent recovery unit to generate a solvent stream and a first acetylene stream; passing the first butadiene stream to a second butadiene separation system to generate a 1,3 butadiene product stream, a heavies stream comprising 1,2 butadiene and C5+ hydrocarbons, and a second acetylene stream; passing the first acetylene stream and the second acetylene stream to a selective hydrogenation reactor system. An embodiment of the invention is one, any or all of prior embodiments in this paragraph up through the second embodiment in this paragraph wherein the selective hydrogenation reactor system comprises passing the acetylene stream and a hydrogen stream to a first selective hydrogenation reactor to generate a first reactor effluent stream with reduced acetylenes; passing the first reactor effluent stream and a second hydrogen stream to a second selective hydrogenation reactor to generate a second reactor effluent stream with reduced acetylenes; and passing a first portion of the second reactor effluent stream and a third hydrogen stream to a third selective hydrogenation reactor to generate a third reactor butadiene stream with less than 100 ppmw acetylenes; wherein the first portion of the second reactor effluent stream is less than 50 wt.-% of the second reactor effluent stream. An embodiment of the invention is one, any or all of prior embodiments in this paragraph up through the second embodiment in this paragraph further comprising passing a second portion of the second reactor effluent stream to the first selective hydrogenation reactor, wherein the second portion of the second reactor effluent stream is greater than 50 wt.-% of the second reactor effluent stream. An embodiment of the invention is one, any or all of prior embodiments in this paragraph up through the second embodiment in this paragraph further comprising passing the third reactor butadiene stream to the butadiene separation system.

Other objects, advantages and applications of the present invention will become apparent to those skilled in the art from the following detailed description and drawings.

### BRIEF DESCRIPTION OF THE DRAWING

The FIGURE is a process for recovering butadiene from a crude C4 process stream.

### DETAILED DESCRIPTION

Butadiene is a major petrochemical commodity used in the production of rubber products such as tires, and other rubber products in the automotive industry. Butadiene needs to have high purity for use in polymer grade rubbers, and should be essentially free of acetylenes, carbonyls, sulfur, and other heavy components.

The use of the UOP KLP™ process provides for the production of 1,3-butadiene from a steam cracked crude C4 stream. The KLP process is a selective hydrogenation process to hydrogenate acetylenes. A solvent extraction process is performed upstream of the hydrogenation process, and the present invention provides for the elimination of one stage of the solvent extraction process, and provides for a more economical process.

The present invention provides a process with several recycle selective hydrogenation reactions in series. The first two reaction stages are operated at higher acetylene concentrations than standard selective hydrogenation designs. This improves the reaction kinetics. The process also does not need to use hydrogen, H2, concentrations in excess of stoichiometric levels to achieve the desired selective hydrogenation of the acetylenes. The reaction recycle effluent stream is used to dilute the acetylene concentrations to reactor target inlet levels. The H2 is limited to control the amount of hydrogenation, and to control the amount of the exotherm of the reactions.

Although not necessarily typical, the reactions can be run to 100% H2 consumption, and this control reduces the risk of exceeding any target maximum exotherms due to over-hydrogenation. A portion of the effluent stream from the last recycle reactor is passed to a final reactor, which operates as a once through reactor for the process stream due to the low acetylene concentration in the feed stream to the last selective hydrogenation reactor. It is more typical to provide excess hydrogen to drive the low product acetylene level.

The present invention is an integrated process for the production of butadiene. The process treats a crude C4 stream by separation of the butadiene and selectively hydrogenating the acetylenes to increase the butadiene production. The crude C4 stream is typically a stream generated in the steam cracking process, with the C4 hydrocarbons separated from the steam cracking effluent stream. The crude C4 stream includes C4 hydrocarbons that comprise butenes, butanes, butadienes, ethylacetylene, vinylacetylene, and methylacetylene. The process, as shown in the FIGURE, includes passing the crude C4 stream 8 to a butadiene separation system 10 to generate a first butadiene stream 12 and an acetylene stream 14. The acetylene stream 14 is passed to a selective hydrogenation reactor system 36 to generate a second butadiene stream 32 and a recycle stream 34. The first butadiene stream 12 is passed to a second butadiene separation system 20.

The second butadiene separation system 20 includes passing the first butadiene stream 12 to a butadiene fractionation unit 28 to generate a 1,3 butadiene overhead stream 26 and a 1,2 butadiene bottoms stream 24. The bottoms stream 24 from the butadiene fractionation unit 28 includes heavies, or compounds having 5 or more carbon atoms that were in the crude C4 stream 8.

The selective hydrogenation reactor system 36 includes passing the acetylene stream 14 and a first hydrogen stream 142 to a first selective hydrogenation reactor 140 to generate a first reactor effluent stream 144 having a reduced acetylene content. The first reactor effluent stream 144 is passed with a second hydrogen stream 152 to a second selective hydrogenation reactor 150 to generate a second reactor effluent stream 154 having a reduced acetylene content. The second reactor effluent stream 154 is divided into a first portion 156 and a second portion 158. The first portion 156 is passed with a third hydrogen stream 162 to a third selective hydrogenation reactor 160 to generate a third reactor butadiene stream 32 with less than 100 ppmw acetylenes. The second portion 158 of the second reactor effluent stream 154 is recycled to the first selective hydrogenation reactor 140. The third reactor butadiene stream 32 is passed to the butadiene separation system 10. In one embodiment, the first portion 156 is less than 50 wt.-% of the second reactor effluent stream. In another embodiment, the first portion is less than 25 wt.-% of the second reactor effluent stream, and in another embodiment, the first portion is less than 15 wt.-% of the second reactor effluent stream. The embodiment wherein the first portion is less than 15 wt.-% of the second reactor effluent stream particularly provides the desired low acetylene in the product. The second portion 158 comprising the difference of first portion from the second reactor effluent stream.

In one embodiment, a reactor 200 is kept off-line, on standby, and can be brought on-line, as one of the other reactors is taken off-line for regeneration.

The butadiene separation system 10 is a solvent extraction system, which includes passing the steam cracked process stream 8, and the third reactor butadiene stream 32, to a first absorption separation column 110, and passing a solvent 184 to the first absorption separation column 110. The first absorption separation column 110 generates a raffinate stream 112 comprising butenes and butanes; a first intermediate stream 114 comprising butadienes, vinylacetylene and ethylacetylene; and a first bottoms stream 116 comprising butadienes, vinylacetylene, ethylacetylene and solvent.

The first intermediate stream 114 is passed to a second absorption separation column 120, and a solvent 186 is also passed to the second absorption separation column 120. The second absorption separation column 120 generates a second overhead stream 122 comprising butadienes, methylacetylene and trace oligomer byproducts such as vinylcyclohexane; and a second bottoms stream 124 comprising acetylenes and solvent. The second overhead stream 122 is passed to a first fractionation column 130 to generate the first butadiene stream 12 and third overhead stream 14. The third overhead stream 14 comprises methyl acetylene and some butadienes. The third overhead stream 14 is passed the selective hydrogenation reactor system 30.

The second bottoms stream 124 from second adsorption separation column 120 is passed to the first adsorption separation column 110. The process further includes passing the first bottoms stream 116 from the first adsorption separation column 110 to a solvent recovery unit 40.

The solvent recovery unit 40 includes passing the first bottoms stream 116 to a first flash separation tank 170. The flash separation tank 170 generates a vapor stream 172 comprising butadienes and acetylenes, and a liquid stream 174 comprising solvent. The solvent will carry some butadienes, acetylenes and other hydrocarbons. The vapor stream 172 is passed to the first adsorption separation column 110, and the liquid stream 174 is passed to a first solvent fractionation column 180. The first solvent fractionation column 180 recovers the solvent in a bottoms stream 182, and generates an overhead stream 188 comprising butadienes and acetylenes, which is passed to the first adsorption separation column 110. The first solvent fractionation column 180 also generates an intermediate stream 187. The intermediate stream 187 is passed to a second solvent fractionation column 190 to strip hydrocarbons from the solvent, and generates an overhead stream 192 comprising acetylenes, and other C4 compounds. The bottoms stream 194 comprises solvent and is returned to the first solvent fractionation column 180. The second solvent fractionation overhead stream 192 is passed to the selective hydrogenation reactor system 36.

The solvent recovery stream 182, or the first solvent fractionation bottoms stream, is split into two portions. A first portion 184 is passed to the first adsorption separation column 110, and the second portion 186 is passed to the second adsorption separation column 120. Preferred solvents include one or more of n-methyl-2-pyrolidone (NMP), dimethylformamide (DMF), acetonitrile, or a mixture of solvents.

In another embodiment of the present invention, the process for generating a butadiene product stream includes passing a steam cracked process stream to a butadiene separation system. The butadiene separation system generates a first butadiene stream and a solvent stream. The solvent stream is passed to a solvent recovery unit, wherein the solvent recovery unit generates a recycled solvent stream and a first acetylene stream. The first butadiene stream is passed to a second butadiene separation system which generates a 1,3 butadiene product stream; a heavies stream comprising C5+ hydrocarbons and 1,2 butadiene; and a second acetylene stream. The first acetylene stream and the second acetylene stream are passed to a selective hydrogenation reactor system. The selective hydrogenation reactor system generates a product stream comprising butadiene, and is low in acetylenes. The product stream is passed to the butadiene separation unit.

The operation of the selective hydrogenation unit includes an acetylene rich stream fed to the selective hydrogenation unit. The acetylene rich stream comprises approximately 30 wt.-% acetylenes in the form of vinyl acetylene and ethylacetylene. This is diluted with a butadiene to maintain the operating region for acetylene concentration to within a safe zone.

The first and second selective hydrogenation reactors in the reactor system or operated with a large recycle, wherein the recycle to feed ratio is greater than 5, and preferably around 7. Recycle can be taken from the first reactor effluent stream or the second reactor effluent stream. The recycle is preferably sufficient to reduce the acetylene concentration to below about 6 wt.-%.

The third reactor in the selective hydrogenation unit will be a much smaller unit than the first or second reactors.

While the invention has been described with what are presently considered the preferred embodiments, it is to be understood that the invention is not limited to the disclosed embodiments, but it is intended to cover various modifications and equivalent arrangements included within the scope of the appended claims.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

What is claimed is:

1. An integrated process for the production of butadiene, comprising:
passing a steam cracked process stream to a butadiene separation system, thereby generating a first butadiene stream and an acetylene stream;
passing the acetylene stream to a selective hydrogenation reactor system, thereby generating a second butadiene stream and a recycle stream; and
passing the first butadiene stream to a second butadiene separation system.

2. The process of claim 1 wherein the second butadiene separation system comprises passing the first butadiene stream to a butadiene fractionation unit to generate a 1,3 butadiene overhead stream and a 1,2 butadiene bottoms stream.

3. The process of claim 2 wherein the 1,2 butadiene bottoms stream comprises 1,2 butadiene and heavies.

4. The process of claim 1 wherein the selective hydrogenation reactor system comprises:
passing the acetylene stream and a hydrogen stream to a first selective hydrogenation reactor to generate a first reactor effluent stream with reduced acetylenes;
passing the first reactor effluent stream and a second hydrogen stream to a second selective hydrogenation reactor to generate a second reactor effluent stream with reduced acetylenes; and
passing a first portion of the second reactor effluent stream and a third hydrogen stream to a third selective hydrogenation reactor to generate a third reactor butadiene stream with less than 100 wppm acetylenes, less than 10 wppm acetylenes, or less than 5 wppm acetylenes.

5. The process of claim 4 further comprising passing a second portion of the second reactor effluent stream to the first selective hydrogenation reactor.

6. The process of claim 4 further comprising passing the third reactor butadiene stream to the butadiene separation system.

7. The process of claim 1 wherein the steam cracked process stream comprises crude C4 hydrocarbons, and the crude C4 hydrocarbons include butenes, butanes, butadienes, ethylacetylene and vinylacetylene.

8. The process of claim 1, further comprising at least one of:
sensing at least one parameter of the process and generating a signal or data from the sensing;
generating and transmitting a signal; or
generating and transmitting data.

9. The process of claim 1 wherein the butadiene separation system comprises:
passing the steam cracked process stream to a first absorption separation column, and passing a solvent to the first absorption separation column, to generate a raffinate stream comprising butenes and butanes; a first intermediate stream comprising butadienes, vinylcyclohexene and ethylacetylene; and a first bottoms stream comprising butadienes, vinylacetylene, ethylacetylene and solvent;
passing the intermediate stream to a second absorption separation column, and passing a solvent to the second absorption separation column, to generate a second overhead stream comprising butadienes, methylacetylene and vinylcyclohexene and pentenes; and a second bottoms stream comprising acetylenes and solvent; and
passing the second overhead stream to a first fractionation column to generate the first butadiene stream and an third overhead stream comprising methyl acetylene and butadiene.

10. The process of claim 9 further comprising passing the third overhead stream to the selective hydrogenation reactor system.

11. The process of claim 9 further comprising passing the second bottoms stream to the first absorption separation column.

12. The process of claim 9 further comprising passing the first bottoms stream to a solvent recovery unit.

13. The process of claim 12 wherein the solvent recovery unit comprises:
passing the first bottoms stream to a first flash separation tank to generate a vapor stream comprising butadienes and acetylenes, and a liquid stream comprising solvent and butadienes and acetylenes;
passing the vapor stream to the first absorption separation column;
passing the liquid stream to a first solvent fractionation column to generate a first solvent fractionation overhead stream; a first solvent fractionation bottoms stream and a first solvent fractionation intermediate stream;
passing the first solvent fractionation intermediate stream to a second solvent fractionation column to generate a second solvent fractionation overhead stream comprising acetylenes, and a second solvent fractionation bottoms stream comprising solvent; and
passing the second solvent fractionation overhead stream to the selective hydrogenation reactor system.

14. The process of claim 13 further comprising passing a first portion of the first solvent fractionation bottoms stream to the first absorption separation column.

15. The process of claim 13 further comprising passing a second portion of the first solvent fractionation bottoms stream to the second absorption separation column.

16. The process of claim 9 wherein the solvent used in the adsorption separation columns is selected from the group consisting of n-methyl-2-pyrolidone (NMP), dimethylformamide (DMF), acetonitrile, and mixtures thereof.

17. A process for the production of butadiene, comprising:
passing a steam cracked process stream to a butadiene separation system, thereby generating a first butadiene stream and a solvent stream;
passing the solvent stream to a solvent recovery unit to generate a solvent stream and a first acetylene stream;
passing the first butadiene stream to a second butadiene separation system to generate a 1,3 butadiene product stream, a heavies stream comprising 1,2 butadiene and C5+ hydrocarbons, and a second acetylene stream;
passing the first acetylene stream and the second acetylene stream to a selective hydrogenation reactor system.

18. The process of claim 17 wherein the selective hydrogenation reactor system comprises:
passing the acetylene stream and a hydrogen stream to a first selective hydrogenation reactor to generate a first reactor effluent stream with reduced acetylenes;
passing the first reactor effluent stream and a second hydrogen stream to a second selective hydrogenation reactor to generate a second reactor effluent stream with reduced acetylenes; and
passing a first portion of the second reactor effluent stream and a third hydrogen stream to a third selective hydrogenation reactor to generate a third reactor butadiene stream with less than 100 ppmw acetylenes; wherein the first portion of the second reactor effluent stream is less than 15 wt-% of the second reactor effluent stream.

19. The process of claim 18 further comprising passing a second portion of the second reactor effluent stream to the first selective hydrogenation reactor, wherein the second portion of the second reactor effluent stream is greater than 50 wt-% of the second reactor effluent stream.

20. The process of claim 18 further comprising passing the third reactor butadiene stream to the butadiene separation system.
